(19) United States
(12) Patent Application Publication
(10) Pub. No.: US 2012/0035519 A1
(43) Pub. Date: Feb. 9, 2012

(54) Trunk bandage or trunk orthosis

(75) Inventor: Frank THELEMANN, BAYREUTH (DE)

(73) Assignee: medi GmbH & Co. KG, Bayreuth (DE)

(21) Appl. No.: 13/186,152

(22) Filed: Jul. 19, 2011

(30) Foreign Application Priority Data
Aug. 5, 2010 (DE) 20 2010 011 083.1

(51) Int. Cl. A61F 5/02

(52) U.S. Cl. 602/19

(57) ABSTRACT

Trunk bandage or trunk orthosis, comprising two connecting portions which are brought together in the stomach or chest region and, to put on the trunk bandage or trunk orthosis, should be made to overlap one another and be connected to one another by way of connecting means, in particular a hook-and-loop fastener, at least one connecting portion being designed as a sheet-like formation, wherein, at the upper or lower end—with respect to the wearing position—, on the connecting portion (6, 7) designed as a sheet-like formation there is formed by way of at least one bending line (10, 11, 12, 13) an edge region (8, 9, 14, 15) which can bend away along this bending line (10, 11, 12, 13) in relation to the remaining region of the connecting portion (16, 17).

The invention relates to a trunk bandage or trunk orthosis, comprising two connecting portions which are brought together in the stomach or chest region and, to put on the trunk bandage or trunk orthosis, should be made to overlap one another and be connected to one another by way of connecting means, in particular a hook-and-loop fastener, at least one connecting portion being designed as a sheet-like formation.

Such trunk bandages or trunk orthoses, for example in the form of lumbar bandages or lumbar orthoses or stabilizing orthoses for the lumbar spine, are used for the treatment of problems in the back region. The patient often feels pain in the back, which he/she attempts to counter by adopting an appropriate posture of the upper body, which is tilted forward or slightly to the side, which in turn leads to further muscle strains and further pain. This can be counteracted by putting on a trunk bandage or trunk orthosis of the type described. In the case of a lumbar bandage, such a trunk bandage or trunk orthosis comprises, for example, a wide, extensive elastic portion, which generally has its greatest width in the back region; widths of 20 cm and more are quite usual. Provided at the two ends of the elastic portion are connecting portions, which are placed one over the other in front of the stomach and connected, for example by way of hook-and-loop fasteners, so that the lumbar bandage can be stretched around the body. The lumbar bandage stabilizes the back and brings about a “lordosis correction”, i.e., as a result of the compression or support caused by the bandage, the pelvis is pushed forward and the spinal column is straightened. Other bandages or orthoses covered by the invention possibly have other aids or contrivances of an elastic or inelastic nature in the back region, all of which however have in common the described connecting means at the front, which may for example also be provided on elastic or inelastic belt portions to be brought in front of the stomach or the chest.

Usually, one of the connecting portions, but mostly both, is/are designed as a sheet-like formation. They directly adjoin the two ends of the elastic portion, which at the ends still has a width of generally 10 cm and more, To allow tensioning around the body, the connecting portions assume the same width, are therefore at least as wide, as the ends of the elastic portion, sometimes are even somewhat wider. The same applies correspondingly in the case of an elastic or inelastic belt or bands on which the connecting portions are arranged. Mostly the two areas, and possibly forms, of the connecting portions are the same, so that they can be placed congruently one over the other. On the connecting portions, the upper and lower ends are often even drawn up or down somewhat, i.e. the width of the connecting portions increases somewhat—with respect to the initial width at the transition to the elastic portion or the belts or bands—and only decreases again toward the end of the connecting portion, in order to achieve overlapping over as large an area as possible.

Sometimes it may happen, in particular when forming widened edge regions, that they press unpleasantly against the stomach during sitting, as a result of the bulging contour of the stomach. This is because the connecting portions have a certain basic stiffness, therefore are to a certain extent flexible, but not elastic, since they are of course intended to produce the firm connection that brings about the therapeutically relevant tensioning in t he elastic region.

The invention consequently addresses the problem of providing a trunk bandage or trunk orthosis that has improved wearing comfort.

To solve this problem, according to the invention it is provided in the case of a trunk bandage or trunk orthosis of the type mentioned at the beginning that, at the upper or lower end—with respect to the wearing position—, on the connecting portion designed as a sheet-like formation there is formed by way of at least one bending line an edge region which can bend away along this bending line in relation to the remaining region of the connecting portion.

In the case of the trunk bandage or trunk orthosis according to the invention, at the upper or lower end, depending on where a potential problem zone is situated, the respective edge region can bend away by way of a defined bending line relatively easily with respect to the remaining region of the connecting portion.

This means that such an edge region can bulge relatively easily during sitting, therefore can bend and thus adapt itself very well to the contour of the stomach, which inevitably changes during sitting. It therefore no longer happens that the edge region stands up on the stomach as a result of its certain stiffness, but rather a simple adaptation, offering pleasant wearing comfort, is possible by “bulging” or “bending away” of the edge region.

For this purpose, the edge region is joined onto the connecting portion in such a way as to allow facilitated bending or pivoting by way of a bending line. According to a development of the invention, this bending line may be realized by way of a weakening of the material, which may also be designed to be relatively small in comparison with the remaining diameter of the material, that is to say thickness of the connecting portion. However, other forms of a bending line are conceivable. The term “bending line” means in this context a relatively sharp line, but may also define an elongate region several millimeters wide, which is likewise understood as a “bending line” in the context according to the invention. As described, stiffening inserts are often incorporated in the enclosing textile material, whether it is a knitted fabric or a terry towelling material, in the connecting portion or, if there are two, both connecting portions. The bending line can then be realized in a simple way, for example by no such stiffening material being incorporated in the region of the bending line, or by this material being made somewhat thinner in this region and the like. Various configurations are conceivable, as long as such an edge region that allows easier bending or bulging can be formed by the connecting portion being designed correspondingly.

With preference, a bending line is respectively provided both at the upper end and at the lower end of the connecting portion, so that an edge region that can bend away is respectively formed both at the upper end and at the lower end. This allows very good adaptation to the shape of the stomach, both in the region of the upper stomach and in the region of the lower stomach, which additionally improves the wearing comfort.

In a development of the invention, both connecting portions may be designed as sheet-like formations, of substantially the same size—and mostly also similar in terms of form—, an edge region that can bend away being respectively formed at the same and on both. If, therefore, both connecting portions are designed as more extensive sheet-like formations, such an edge region that can bend away is, for example, respectively located on each sheet-like formation, at the lower end or at the upper end or at both ends, in each case defined by way of a bending line. This has the effect that ultimately the edge regions of both connecting portions, which in the wearing position are arranged congruently over one another, have sufficient flexibility, and can bend away together and can thus adapt themselves to the contour of the stomach. With preference, corresponding edge regions are defined both at the upper end and at the lower end.

Such a bending line expediently extends substantially over the entire length of a connecting portion, in order to ensure that the edge region can bend away over as large an area as possible, or over the entire length, of the connecting portion, which is significantly stiffer in comparison with the adjoining elastic or inelastic portions or belts. The length of the respective bending line ultimately depends on the form or geometry of the respective connecting portion, and should be chosen correspondingly.

Further advantages, features and details of the invention emerge from the exemplary embodiment described below and from the drawings, in which:

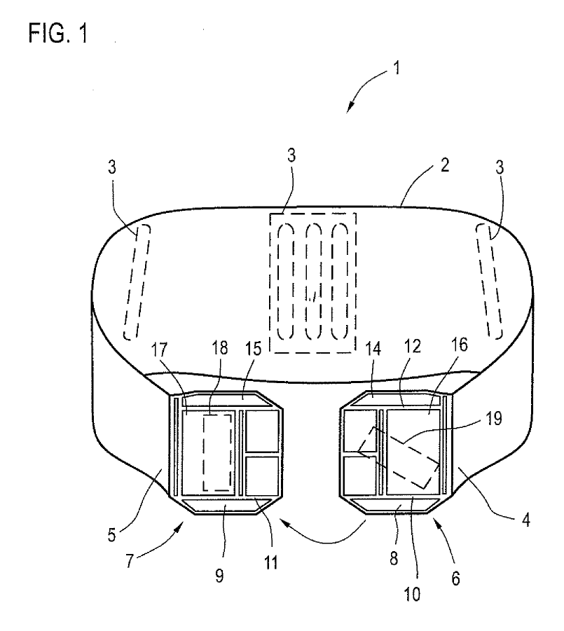

FIG. 1 shows a perspective view of a trunk bandage or trunk orthosis according to the invention in the form of a lumbar bandage in the state in which it has not been put on,

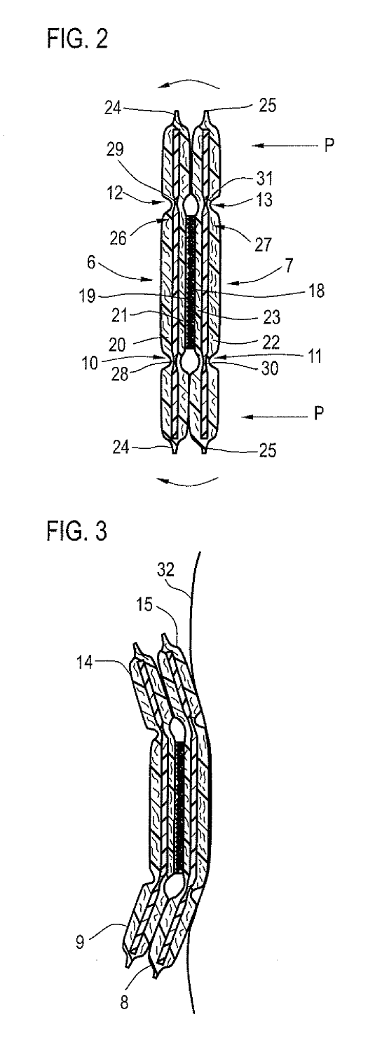

FIG. 2 shows a sectional view through the two connecting portions, overlapping one another in the wearing position, of the lumbar bandage from FIG. 1 without deformation, and

FIG. 3 shows the view from FIG. 3 with upper and lower edge regions bent away.

FIG. 1 shows a lumbar bandage 1 according to the invention, comprising an elastic portion 2, which is formed from a knitted fabric, in which elastic threads, that is to say rubber threads and the like, are incorporated, in order to allow elasticity at least in the circumferential direction, but with preference also in the vertical direction, i.e. to allow the elastic portion 2 to be stretched and tensioned firmly around the upper body. Only indicated by dashed lines are various supporting elements 3, for example plastic struts, which are incorporated among or in the woven fabric or placed on it and, as described, are either covered over by the knitted fabric or else, if they are placed on the knitted fabric, are covered with a corresponding textile layer. The basic structure of such elastic portions of bandages of this type is sufficiently well-known.

Attached, for example adhesively attached, to the two ends 4, 5 of the elastic portion in band form are two extensive connecting portions 6, 7, which can be achieved using suitable adhesive threads and the like, which when heated achieve a firm adhesive bond between the adjacent fabrics. As FIG. 1 shows, the two connecting portions 6, 7 are relatively extensive, and of a similar form. The width of the connecting portions—with respect to the vertical—corresponds at least to the width of the ends 4, 5 of the elastic portion 2. As can be seen, however, in the lower region, the width of the connecting portions 6, 7 increases somewhat, so that there form as it were two downwardly drawn edge regions 8, 9. In the example shown, these are approximately trapezoidal, but they may also have a round edge contour and the like.

By way of appropriate bending lines 10, 11 and 12, 13, the corresponding edge portions 8, 9, 14, 15 are then defined on the connecting portions 6, 7, that is to say the sheet-like formations, as edge portions that can bend away, 8, 9 at the lower end of the connecting portion and 14, 15 at the upper end of the connecting portion. This means that these edge portions 8, 9, 14, 15 can bend away relatively easily with respect to the remaining regions of the connecting portion 16, 17 along or around this bending line 10, 11, 12, 13. This allows a simple adaptation of the form of the edge regions of the connecting portions 6, 7 to the shape of the wearer's stomach, which will be discussed in more detail below.

To put on the lumbar bandage 1, it is placed behind the wearer's back and brought to the front. The connecting portion 7 is placed on the side of the stomach, after which the connecting portion 6 is placed over the connecting portion 7 and the two are firmly connected to one another by way of suitable connecting means 18, 19, here for example a hook-and-loop fastener. The connection must be firm in order to maintain the desired tensioning that is produced by pulling the elastic portion 2 into the wearing position and is intended to act on the body.

Consequently, in the wearing position, the two connecting portions 6, 7 lie one over the other, as FIG. 2 shows. In FIG. 2, a sectional view through the two connecting portions 6, 7 lying one over the other is represented, also clearly revealing the inner structure of the connecting portions 6, 7. Each connecting portion 6, 7 comprises an outer and an inner textile layer 20, 21 and 22, 23, respectively. The respectively inner textile layer 21, 23 may be, for example, a knitted fabric, the outer textile layer 20, 22 may be, for example, a terry towelling textile. At the edges, the two textile layers 20, 21 and 22, 23 are connected to one another by way of adhesive connections 24, 25, which applies to the entire peripheral extent. The corresponding connecting means 18 and 19, which are connected to one another, are also provided on the inner textile layers 21, 23.

To stiffen the respective connecting portions 6, 7, incorporated in each case between the respective textile layers 20, 21 and 22, 23 is a stiffening layer 26, 27 in panel form, for example of a rubber or plastics material, which although it has a certain flexibility is not elastic. To form the bending lines 10, 11, 12, 13, on these layers 26, 27 there is then formed in each case a weakening of the material 28, 29, 30, 31, i.e. the thickness of the layers 26, 27 in these regions defining the bending lines is reduced, as can be clearly seen in the sectional representation according to FIG. 2. This is achieved in a simple way in that, after introducing the layers 26, 27 between the textile layers 20, 21 and 22, 23, a material composite in which the edges are fused together to establish the bonds 24, 25 is produced. At the same time, by local heating along the bending lines 10, 11, 12, 13, in this region too the textile layers 20, 21 are connected to the layer 26 and the textile layers 22, 23 are connected to the layer 27 by beginning to melt, and the respective layer 26, 27 is also locally heated by the introduction of heat and weakening of the material is brought about by the application of pressure, that is therefore to reduce the diameter in the heated region. In this way, therefore, the text layers and the intermediate layer are connected to one another, and at the same time the respective bending lines are also defined by the weakenings of material 28, 29, 30, 31 being formed. Consequently, altogether a structure of the connecting portions such as that shown in section in FIG. 2 is obtained.

The form and alignment of the connecting portions 6, 7 that are shown in FIG. 2 are ultimately obtained when the wearer has put on the lumbar bandage 1 and is standing, so in the stomach region there is therefore no sitting-induced bulging of the stomach. If the wearer then sits down, there are inevitably displacements of the fabric in the stomach region, which bulges (cf. FIG. 3). When this happens, a pressure P. see FIG. 2, is exerted from the inner side onto the overlapping connecting portions 6, 7, but in particular in the edge regions 8, Sand 14, 15, as represented in FIG. 2 by the two arrows indicated by P. However, since these edge regions 8, 9, 14, 15 are designed such that they can bend away in relation to the respective remaining regions of the connecting portions 16 and 17 by way of the bending lines 10, 11 and 12, 13, it then happens that the two edge regions 8, 9 and 14, 15 lying one over the other yield to the pressure P and bulge outward, as shown in FIG. 3. This means that they bend about the respective bending lines 10, 11 and 12, 13 and assume a bent-away position, angled from the original, elongate position in relation to the regions of the connecting portions 16, 17, as FIG. 3 shows. In FIG. 3, the stomach 32 is represented, so that it can be seen that the bending-away edge regions 8, 9 and 14, 15 are deformed in relation to the remaining regions of the connecting portions 16, 17 in a manner following the shape of the stomach or hugging it. This is much more pleasant for the wearer, since it has the effect that they no longer stand up on the stomach as a result of their stiffness and cause rubbing or pressing of the edges into the stomach 32.

Even though the exemplary embodiment described shows two extensive connecting portions with respective edge regions that can bend away 8, 9 and 14, 15, it is of course also conceivable, for example, to design only the two lower edge portions 8, 9 on the two connecting portions 6, 7 such that they can bend away, or only the two upper edge portions 14, 15. Furthermore, it is conceivable to make only one of the connecting portions 6, 7 so extensive and provide one or both edge portions that can bend away, while the other connecting portion is much narrower, no that therefore its width, seen vertically, is much less than that of the other connecting portion and, as a result of this, it does not have to have an edge region that can bend away.

In the case of bandages or orthoses of a different structure, the connecting portions may also be provided on elastic or inelastic belts or bands to be brought in front of the stomach or the chest.

1. Trunk bandage or trunk orthosis, comprising two connecting portions which are brought together in the stomach or chest region and, to put on the trunk bandage or trunk orthosis, should be made to overlap one another and be connected to one another by way of connecting means, in particular a hook-and-loop fastener, at least one connecting portion being designed as a sheet-like formation, characterized in that, at the upper or lower end—with respect to the wearing position—, on the connecting portion designed as a sheet-like formation there is formed by way of at least one bending line an edge region which can bend away along this bending line in relation to the remaining region of the connecting portion.

2. Trunk bandage or trunk orthosis according to claim 1, characterized in that an edge region that can bend away is respectively formed both at the upper end and at the lower end by way of in each case a bending line.

3. Trunk bandage or trunk orthosis according to claim 1, characterized in that a bending line is realized by means of a weakening of the material.

4. Trunk bandage or trunk orthosis according to claim 1, characterized in that both connecting portions are designed as sheet-like formations, of substantially the same size, an edge region that can bend away being respectively formed at the same end on both.

5. Trunk bandage or trunk orthosis according to claim 1, characterized in that a bending line extends substantially over the entire length of a connecting portion.
